(19) United States
(12) Patent Application Publication
(10) Pub. No.: US 2018/0280483 A1
(43) Pub. Date: Oct. 4, 2018

(54) AGENT FOR PROTECTING UPPER RESPIRATORY TRACT AND FOOD OR DRINK COMPOSITION FOR PROTECTING UPPER RESPIRATORY TRACT

(71) Applicant: MORINAGA MILK INDUSTRY CO., LTD., Tokyo (JP)

(72) Inventors: Kouichirou SHIN, Zama-shi (JP); Hiroyuki WAKABAYASHI, Zama-shi (JP); Masahiko KUROKAWA, Nobeoka-shi (JP)

(73) Assignee: MORINAGA MILK INDUSTRY CO., LTD., Tokyo (JP)

(21) Appl. No.: 15/753,727

(22) PCT Filed: Jul. 15, 2016
(86) PCT No.: PCT/JP2016/070985

§ 371 Date: Feb. 20, 2018

(30) Foreign Application Priority Data
Aug. 21, 2015 (JP) 2015-163972
Feb. 25, 2016 (JP) 2016-034419

(51) Int. Cl. A61K 38/40; A61K 38/44; A61K 31/7004; A61K 31/198; A61K 31/194; A61P 11/04; A23L 33/17

(52) U.S. Cl. CPC A61K 38/40; A61K 38/443; A61K 31/7004; A61K 31/198; A61K 31/194; A23V 2250/61; A23L 33/17; A23V 2002/00; A23V 2200/314; A23V 2250/54248; A23V 2250/5425; A61P 11/04

(57) ABSTRACT

An agent for protecting upper respiratory tracts or a food or drink composition for protecting upper respiratory tracts which is effective in ameliorating various symptoms relating to upper respiratory tracts is provided.

An agent for protecting upper respiratory tracts and a food or drink composition for protecting upper respiratory tracts containing lactoferrin, lactoperoxidase, glucose oxidase, glucose source, and a pH adjusting component as active ingredients is provided.

The agent for protecting an upper respiratory tract according to the present invention and the food or drink composition for protecting an upper respiratory tract can ameliorate various symptoms relating to upper respiratory tracts.

### TECHNICAL FIELD

The present invention relates to an agent for protecting upper respiratory tracts or a food or drink composition for protecting upper respiratory tracts containing lactoferrin, lactoperoxidase, glucose oxidase, glucose source, and a pH adjusting component as active ingredients.

### BACKGROUND ART

Dryness of an upper respiratory tract mucosa (hereinafter referred to as “upper respiratory tract mucosal dryness”) is a sensation triggered by a dried mucosa of an upper respiratory tract (oral cavity, pharynx, and larynx), and known as a typical symptom of xerostomia (dry mouth) or Sjogren's syndrome. The upper respiratory tract mucosal dryness is also perceived as a sensation complicated with thirst in many cases. In order to ameliorate such upper respiratory tract mucosal dryness, a method employing artificial saliva having a composition analogous to that of human saliva which is sprayed into the oral cavity or use of an agent which promotes salivation are known.

Also when the throat develops an inflammation which then irritates a nerve, a sensation so called “scratchy throat” occurs. This scratchy throat is a sensation caused by an inflammation of the throat due to smoking, excessive vocalization, reflux esophagitis, stress, allergy, and the like. Such a sensation is ameliorated generally by a practice such as gargle or using a throat lozenge.

Patent Document 1 discloses an intraoral plaster preparation whose active ingredient is lactoferrin, lactoperoxidase, or lysozyme, and the like. This oral cavity plaster is described to enable a therapy via inducing salivation, ameliorating pain in the oral cavity or the throat, anti-inflammatory effects on oral cavity or upper respiratory tract and esophagus, and the like.

Patent Document 2 also discloses a method for producing a powder composition whose active ingredient is at least one selected from lactoferrin, lactoperoxidase, conalbumin, and lysozyme. This powder composition is described to be capable of treat xerostomia or upper respiratory tract inflammation.

Patent Document 3 also discloses a bactericidal agent for use in the oral cavity containing lactoperoxidase, glucose oxidase, glucose, and a pH adjusting component. This bactericidal agent for use in the oral cavity is described to be capable of sterilizing bacteria in the oral cavity effectively and also be effective in prophylaxis and/or therapy of diseases caused by the bacteria in the oral cavity.

Meanwhile, smoking is known to have various effects on diverse organs in a human including primarily diseases in the respiratory system. For example in the oral cavity which is exposed to the inspired gas (cigarette smoke; mainstream smoke) firstly upon smoking, various symptoms such as coloring of tooth surface or gingiva, odor (ozostomia), tartar deposition, periodontal disease, lingua nigra, leukoplakia, necrotizing sialometaplasia, oral cavity cancer are reported to be triggered by the smoking (Non-Patent Document 1).

The smoking is also reported to induce upper respiratory tract mucosal dryness or thirst (Non-Patent Documents 2 and 3). “Scratchy throat” is also known to be experienced by many smokers. This is believed to be caused partly by dryness of mucosa of the oral cavity, pharynx, and larynx due to the heat of the smoke inspired upon smoking together with inflammation caused by hazardous components contained in the smoke which irritate the mucosa of the oral cavity, pharynx, and larynx.

Based on the understandings described above, it is believed that smokers are suffering from multiple discomforts including the upper respiratory tract mucosal dryness, thirst, and scratchy throat.

Such symptoms are the symptoms specific to the smokers, and the agent and the compositions disclosed in the aforementioned Patent Documents 1 and 2 are not successful in exerting sufficient effects because of no consideration of such smoker-specific symptoms. It is a matter of course that the problems are solved entirely by quitting smoking, and there is no any other known means for convenient inhibition of the multiple discomforts such as the upper respiratory tract mucosal dryness, thirst, and scratchy throat associated with the smoking by means of reducing the smoking-induced irritations to the mucosa of the oral cavity, pharynx, and larynx.

Accordingly, an effective and convenient means for ameliorating the multiple discomforts such as the upper respiratory tract mucosal dryness, thirst, and scratchy throat associated with smoking has been desired.

A cold exhibiting symptom such as sneeze, nasal discharge, fever, fatigue is known to be induced mostly by upper respiratory tract infections with various viruses. Its prophylaxis and symptom amelioration are attempted by known means such as preservation of body resistance by health control via sufficient sleeping and overwork prevention as well as sanitary procedure such as gargle and mask-wearing for ensuring cleanness of the oral cavity and moistening the upper respiratory tracts (Non-Patent Documents 5 and 6).

Nevertheless, a further development of the means for ameliorating the cold symptoms has been desired.

### CITATION LIST

### Patent Literature

Patent Document 1: JP-A No.2002-322088
Patent Document 2: JP-A No.2003-137809
Patent Document 3: WO No. 2008/105113 pamphlet

### Non-Patent Literature

Non-Patent Document 1: Nobuo Yoshizawa, cardiologist, Journal of JCS Cardiologists: 2004, Vol.12, No.2, pp.351-356
Non-Patent Document 2: Maryam Rad et al., J.Dent.Res., Dent.Clin., Dent.Prospects, 2010, Vol.4, No.4, pp.110-114
Non-Patent Document 3: Shyuji Sawaki, oral cavity•pharynx department, 1990, Vol.2, No.2, pp.47-50
Non-Patent Document 4: Aleksandra Konic-Risticet al., J.Med.Food, 2015, Vol.18, No.4, pp.483⁻488
Non-Patent Document 5: Emiko Kono et al., JAPAN SOCIETY OF STOMATO-PHARYNGOLOGY, 2003, Vol.15, No.2, pp.199-207
Non-Patent Document 6: Masahiro Kawamoto et al., JAPAN SOCIETY OF STOMATO-PHARYNOGOLOGY, 2011, Vol.24, No.2, pp.129-133

### SUMMARY OF THE INVENTION

### Technical Problem

The present invention was established in view of the circumstance described above, and its object is to provide an agent for protecting upper respiratory tracts or a food or drink composition for protecting upper respiratory tracts which is effective to ameliorate various symptoms associated with an upper respiratory tract.

### Solution to Problem

As a result of our intensive study, the inventors discovered that components including lactoferrin, lactoperoxidase, glucose oxidase, glucose source, and a pH adjusting component are effective in ameliorating various symptoms associated with upper respiratory tracts, and established the present invention.

Thus, the present invention is an agent for protecting an upper respiratory tract containing lactoferrin, lactoperoxidase, glucose oxidase, glucose source, and a pH adjusting component as active ingredients.

The present invention allows the aforementioned upper respiratory tract protection to be based on at least one selected from removal of foreign bodies from the upper respiratory tract, prevention of foreign bodies from entering the upper respiratory tract, moisturization of the upper respiratory tract, inhibition of the dryness of the mucosa of the upper respiratory tract, and reduction in the irritation of the mucosa of the upper respiratory tract.

The present invention allows the aforementioned upper respiratory tract protection to be prophylaxis and/or therapy of cold symptoms. In such a case, the aforementioned cold symptoms may be one or more symptoms selected from the group consisting of fever, sore throat, cough, nasal discharge, nasal congestion, phlegm, headache, joint pain, and muscle pain. Especially, the aforementioned cold symptoms may also be a symptom of a high body temperature of 38° C. or higher.

The present invention can be used by smokers. In such a case, the use before smoking is intended especially.

In the present invention, the aforementioned upper respiratory tract may be pharynx or larynx.

In the present invention, the aforementioned pH adjusting component may be an organic acid and/or a salt of the organic acid. In such a case, the aforementioned organic acid may especially be one or more organic acids selected from the group consisting of citric acid, lactic acid, malic acid, succinic acid, tartaric acid, and glutamic acid.

Furthermore, the present invention is a food or drink composition for protecting an upper respiratory tract containing lactoferrin, lactoperoxidase, glucose oxidase, glucose source, and a pH adjusting component as active ingredients.

In addition, the present invention is use of lactoferrin, lactoperoxidase, glucose oxidase, glucose source, and a pH adjusting component for an agent for protecting an upper respiratory tract.

Moreover, the present invention is use of lactoferrin, lactoperoxidase, glucose oxidase, glucose source, and a pH adjusting component for a food or drink composition for protecting an upper respiratory tract.

Furthermore, the present invention is use of lactoferrin, lactoperoxidase, glucose oxidase, glucose source, and a pH adjusting component for producing an agent for protecting an upper respiratory tract.

In addition, the present invention is use of lactoferrin, lactoperoxidase, glucose oxidase, glucose source, and a pH adjusting component for producing a food or drink composition for protecting an upper respiratory tract.

Moreover, the present invention is use of lactoferrin, lactoperoxidase, glucose oxidase, glucose source, and a pH adjusting component in an agent for protecting an upper respiratory tract.

Furthermore, the present invention is use of lactoferrin, lactoperoxidase, glucose oxidase, glucose source, and a pH adjusting component in a food or drink composition for protecting an upper respiratory tract.

In addition, the present invention is lactoferrin, lactoperoxidase, glucose oxidase, glucose source, and a pH adjusting component for use in prophylaxis, amelioration, and/or therapy of an upper respiratory tract disorder.

Moreover, the present invention is a method for prophylaxis, amelioration, and/or therapy of an upper respiratory tract disorder using lactoferrin, lactoperoxidase, glucose oxidase, glucose source, and a pH adjusting component as active ingredients.

### Advantageous Effects of Invention

The present invention is effective in ameliorating various symptoms relating to upper respiratory tracts.

Typically, the present invention has effects to reduce the stimulation by smoke to the mucosa of the oral cavity, pharynx, and larynx of a smoker thereby inhibiting the dryness of the mucosa and to inhibit discomforts during smoking such as upper respiratory tract mucosal dryness, thirst, and scratchy throat. Furthermore, the present invention has a reduced side effect even if given for a prolonged period, thereby enabling a convenient administration.

The present invention also has effects to suppress cold symptoms development, number of symptomatic days, and exacerbation to a serious disease via protection of upper respiratory tracts. In addition, the present invention allows the ingestion volume and the number of ingestion times to be selected arbitrarily because of its reduced side effect and high safety, thereby enabling a long term routine ingestion.

### DESCRIPTION OF EMBODIMENTS

Preferred embodiments of the present invention are described below in detail. Nevertheless, the present invention is not limited to the following preferred embodiments and should be understood to be changed arbitrarily within the scope of the invention. Unless otherwise specified, the percent is represented by mass in this specification.

### <Lactoferrin>

Lactoferrin is one of milk proteins which is a non-hazardous and naturally-occurring iron-binding protein (capable of binding to 2 iron ions per molecule) which is contained also in mammalian milk as well as secretions such as saliva, tear, and airway mucus.

The lactoferrin employed in the present invention can be obtained from milk of mammals such as humans, cows, horses, sheep, and goats, and may be any of those produced by general methods. For example, those produced by the method described below starting from a skimmed milk obtained by removing fats from cow's milk are employed.

Thus, an ion exchanger (for example, trade name: CM-SepharoseFF, manufactured by Amersham Pharmacia) is packed into a column which is then washed with water to equilibrate the ion exchanger, and thereafter a skimmed milk cooled to 4° C. is passed through the column and the effluent is recovered and then passed through the column again. Then distilled water is passed through the column, and saline is passed through the column thereby obtaining an eluate of basic proteins once adsorbed to the ion exchanger.

The eluate thus obtained is ultrafiltrated using an ultrafiltration module (for example, trade name: SLP0053, manufactured by Asahi Kasei Corporation). Thereafter, water is added and a diafiltration is conducted using the same device for desalting, followed by lyophilization thereby obtaining bovine lactoferrin in the form of a powder.

By the method described above, bovine lactoferrin whose purity is 95% by mass or more can be obtained.

The purity of the lactoferrin can be measured by known methods such as liquid chromatography and electrophoresis.

It is also possible in the present invention to use a lactoferrin-containing fluid in each purification step before lyophilization.

### <Lactoperoxidase>

Lactoperoxidase is one of milk proteins which is oxidoreductase contained also in mammalian milk as well as secretions such as saliva, tear, and airway mucus, and can be purified industrially from cow's milk.

The lactoperoxidase employed in the present invention can be obtained from mammalian milk, for example the milk of humans, cows, horses, sheep, and goats. For example as the method disclosed in Japanese Unexamined Patent Application Publication No.5-41981, an industrial production starting from a non-heated whey or a skimmed milk according to general methods (for example, ion exchange chromatography) is preferable, and it is also possible to use commercially available lactoperoxidases derived from natural products (for example manufactured by Biopole) or a recombinant lactoperoxidase [for example a recombinant lactoperoxidase expressed and purified by the method by Xin et al., (Biochemical and Biophysical Research Communications, 2000, Vol.271, pp.831-836) or commercially available recombinant lactoperoxidases].

### <Glucose Oxidase>

Glucose oxidase employed in the present invention are commercially-available glucose oxidases (for example, manufactured by SHINNIHON CHEMICALS Corporation) which are enzymes produced by microorganisms such as Aspergillus niger and Penicillium chrysogenum.

### <Glucose Source>

The glucose source employed in the present invention may for example be commercially-available glucoses for food additives (for example, manufactured by NIHON SHOKUHIN KAKO CO., LTD.).

### <pH Adjusting Component>

In the present invention, the pH is adjusted stably by using a pH adjusting component. The pH adjusting component may be any of those having buffering capacities for adjusting the pH, and a preferred example is an organic acid and/or a salt of the organic acid, and a more preferred example is one or more organic acids selected from the group consisting of commercially available food additives such as citric acid, lactic acid, malic acid, succinic acid, tartaric acid, and glutamic acid, as well as one or more salts selected from the group consisting of citrates (for example, trisodium citrate, tripotassium citrate), lactates (for example, sodium lactate), malates (for example, sodium malate), succinates (for example, monosodium succinate, disodium succinate), tartarates (for example, sodium tartarate, potassium hydrogen tartarate), glutamates (for example, sodium glutamate, potassium glutamate).

### <Agent for Protecting an Upper Respiratory Tract>

The agent for protecting an upper respiratory tract according to the present invention contains lactoferrin, lactoperoxidase, glucose oxidase, glucose source, and a pH adjusting component as active ingredients.

The amount of each active ingredient in the agent for protecting an upper respiratory tract according to the present invention can appropriately be selected on the basis of the dosage forms, for example, as described below.

Thus, as the amount of lactoferrin contained in the agent for protecting an upper respiratory tract according to the present invention, 0.001% by mass or more is preferable, 0.01% by mass or more is more preferable, 0.1% by mass or more is especially preferable. While no upper limit is specified, 5% by mass or less, preferably 10% by mass or less is exemplified.

As the amount of lactoperoxidase contained in the agent for protecting an upper respiratory tract according to the present invention, 0.0001% by mass or more is preferable, 0.001% by mass or more is more preferable. While no upper limit is specified, 0.05% by mass or less, preferably 0.1% by mass or less is exemplified.

As the amount of glucose oxidase contained in the agent for protecting an upper respiratory tract according to the present invention, 0.001% by mass or more is preferable, 0.01% by mass or more is more preferable. While no upper limit is specified, 0.5% by mass or less, preferably 1.0% by mass or less is exemplified.

As the amount of glucose source contained in the agent for protecting an upper respiratory tract according to the present invention, 0.01% by mass or more is preferable, 0.1% by mass or more is more preferable. While no upper limit is specified, 5% by mass or less, preferably 10% by mass or less is exemplified.

As the amount of a pH adjusting component contained in the agent for protecting an upper respiratory tract according to the present invention, 0.01% by mass or more is preferable, 0.1% by mass or more is more preferable. While no upper limit is specified, 8% by mass or less, preferably 15% by mass or less is exemplified.

The agent for protecting an upper respiratory tract according to the present invention preferably exhibits, when suspended in a solvent such as saliva, a weakly acidic pH. Such a weakly acidic pH is adjusted by the aforementioned pH adjusting agent preferably at pH4.4 to 5.9, more preferably at pH4.4 to 5.4.

As used herein, “upper respiratory tract protection” can for example be based on at least one selected from removal of foreign bodies from the upper respiratory tract, prevention of foreign bodies from entering the upper respiratory tract, moisturization of the upper respiratory tract, inhibition of the dryness of the mucosa of the upper respiratory tract, and reduction in the irritation of the mucosa of the upper respiratory tract. In the present invention, it is based most preferably on at least one selected from moisturization of the upper respiratory tract, inhibition of the dryness of the mucosa of the upper respiratory tract, and reduction in the irritation of the mucosa of the upper respiratory tract.

Also as used herein, “upper respiratory tract disorder” may for example be invasion of foreign bodies into the upper respiratory tract, dryness of the mucosa of the upper respiratory tract, cold symptoms, sore throat (including cases resulting from cold symptoms). Based on the results described below, the active ingredient constituting the agent for protecting an upper respiratory tract according to the present invention can be used in prophylaxis, amelioration, and/or therapy of the upper respiratory tract disorder, and can be used also in methods for prophylaxis, amelioration, and/or therapy of the upper respiratory tract disorder.

Also in the present invention, this upper respiratory tract protection may be prophylaxis and/or therapy of cold symptoms such as fever, sore throat, cough, nasal discharge, nasal congestion, phlegm, headache, as well as joint pain and/or muscle pain. In the present invention, it is especially preferred to target the fever as this cold symptoms.

As used herein, “fever” refers especially to a symptom of a high body temperature of 38° C. or higher.

As used herein, “upper respiratory tract mucosa” includes not only the so-called mucosa in an oral cavity but also pharyngeal and laryngeal mucosa. As used herein, “upper respiratory tract mucosa dryness-inhibiting effect” refers, for example, to an effect to increase the mouth wetness of a smoker thereby inhibiting thirst and scratchy throat. This may be based on an effect to inhibit the dryness of the mucosa via reduction in the irritation by smoke to the mucosa in the oral cavity, pharynx, and larynx during smoking. The agent for protecting an upper respiratory tract according to the present invention is employed preferably by smokers because of its inhibition especially of the pharyngeal and laryngeal mucosa.

The agent for protecting an upper respiratory tract according to the present invention has an effect to inhibit or reduce the dryness of the mucosa via reduction in the irritation by smoke to the mucosa in the oral cavity, pharynx, and larynx of a smoker when used either before smoking or after smoking, and it is used by the smoker most preferably before smoking from the prophylactic point of view.

While the “upper respiratory tract” generally includes nose, nasal cavity, nasopharynx, pharynx, and larynx among respiratory organs (airways), it is especially preferred that the pharynx or larynx is subjected because the effect of the invention can readily be achieved.

### <Medicament and Quasi-Drug>

The agent for protecting an upper respiratory tract according to the present invention is employed in the form of a medicament or a quasi-drug (hereinafter referred to as “medicament of the present invention”), and is given to a human preferably via oral administration.

The medicament of the present invention is used preferably in order to reduce the stimulation by smoke to the mucosa of the oral cavity, pharynx, and larynx of a smoker thereby inhibiting the dryness of the mucosa, and to inhibit discomforts such as upper respiratory tract mucosal dryness, thirst, and scratchy throat during smoking.

It is also preferable to use the medicament of the present invention for the purpose of prophylaxis and/or therapy of cold symptoms (especially fever).

Since the medicament of the present invention is given preferably via oral administration, the form of the formulation is given as a dosage form capable of being administered orally such as tablets and troches.

When the agent for protecting an upper respiratory tract according to the present invention is used in the form of a medicament, the timing of the administration is not limited particularly and can appropriately be selected depending on the symptom to be subjected, but the administration either before or after smoking is preferable, and administration during the period between 10 minutes before and after smoking is more preferable. In particular, use before smoking is preferable because the discomforts experienced by the smoker such as upper respiratory tract mucosal dryness, thirst, and scratchy throat can effectively be inhibited.

While the dose of the medicament of the present invention may vary depending on the experience of smoking, the degrees of cold symptoms, the form of the formulation, the age of the subject to be administered, it is preferably 10 mg to 10 g per one administration. The administrations frequency can appropriately be selected depending on the smoking frequency and the degrees of the cold symptoms. It is preferably be equivalent to the smoking frequency when the subject is a smoker.

As long as the effect of the present invention is not affected adversely, the medicament of the present invention may contain other components having effects to inhibit discomforts such as upper respiratory tract mucosal dryness, thirst, and scratchy throat during smoking and other components having prophylactic and/or therapeutic effects on cold symptoms.

The medicament of the present invention may contain additives employed widely in medicaments. Examples of the additives include excipients, binders, disintegrants, lubricants, stabilizers, flavoring agents.

Examples of the excipients include saccharide derivatives such as lactose, sucrose, glucose, mannitol, and sorbitol; starch derivatives such as corn starch, potato starch, cc-starch, dextrin, and carboxymethyl starch; a cellulose derivative such as crystalline cellulose, hydroxypropyl cellulose, hydroxypropylmethyl cellulose, carboxymethyl cellulose, and calcium carboxymethyl cellulose; gum arabic; dextran; pullulan; silicate derivatives such as light silicic anhydride, synthetic aluminum silicate, and magnesium aluminometasilicate; phosphate derivatives such as calcium phosphate; carbonate derivatives such as calcium carbonate; and sulfate derivatives such as calcium sulfate.

Examples of the binders include gelatin; polyvinyl pyrrolidone; and Macrogol.

Examples of the disintegrants include chemically modified starches or cellulose derivatives such as croscarmellose sodium, sodium carboxymethyl starch, and crosslinked polyvinylpyrrolidone.

Examples of the lubricants include talc; stearic acid; metal stearates such as calcium stearate and magnesium stearate; colloidal silica; waxes such as veegum and spermaceti; boric acid; glycol; carboxylic acids such as fumaric acid and adipic acid; sodium carboxylates such as sodium benzoate; sulfates such as sodium sulfate; leucine; lauryl sulfates such as sodium lauryl sulfate and magnesium lauryl sulfate; silicic acids such as silicic anhydride and silicic hydrate; and starch derivatives.

Examples of the stabilizers include a p-hydroxybenzoate such as methyl paraben and propyl paraben; alcohols such as chlorobutanol, benzyl alcohol, and phenylethyl alcohol; benzalkonium chloride; acetic anhydride; and sorbic acid.

Examples of the flavoring agents include sweeteners, acidifiers, and flavors.

The medicament of the present invention can be produced for example by formulating lactoferrin, lactoperoxidase, glucose oxidase, glucose source, and a pH adjusting component together with the aforementioned additives.

### <Food or Drink Composition>

Lactoferrin, lactoperoxidase, glucose oxidase, glucose source, and a pH adjusting component which are active ingredients in the present invention can be given in the form of a food or drink composition, which is preferred in that the upper respiratory tract mucosal dryness, discomforts such as thirst and scratchy throat associated with smoking are inhibited and in that the use such as cold symptoms remedy is realized, both by ingesting the relevant food or drink composition.

Thus the present invention is a food or drink composition for protecting an upper respiratory tract containing lactoferrin, lactoperoxidase, glucose oxidase, glucose source, and a pH adjusting component as active ingredients (hereinafter referred to as “food or drink composition according to the invention”).

The food or drink composition according to the invention contains the lactoferrin, the lactoperoxidase, the glucose oxidase, the glucose source, and the pH adjusting component as active ingredients capable of inhibiting the upper respiratory tract mucosal dryness, discomforts such as thirst and scratchy throat associated with smoking or active ingredients enabling prophylaxis and/or therapy of the cold symptoms.

Thus, the form and the physical state is not limited particularly as long as the upper respiratory tract protection effects are not affected adversely and as long as oral ingestion is possible, and the production is possible using starting materials used for an ordinary food or drink by an ordinary method except for allowing the lactoferrin, the lactoperoxidase, the glucose oxidase, the glucose source, and the pH adjusting component to be contained.

The aforementioned food and drink products, regardless of their forms such as liquids, pastes, gelling solids, and powders, may for example be tablet confectioneries and liquid diets, as well as flour products such as breads, macaronis, spaghetties, noodles, cake mixes, deep frying flours, and bread crumbs; instant foods such as instant noodles, cup-contained instant noodles, retort-cooked foods, cooked and canned foods, microwaving foods, instant soups or stews, instant miso soups or clear soups, canned soups, freeze-dried foods, and other instant foods; processed agricultural products such as canned agricultural products, canned fruits, jams or marmalades, pickles, boiled beans, agricultural dry foods, cereals (processed grain products); processed marine products such as canned marine products, fish meat hams and sausages, marine paste products, marine delicacies, and cocked and seasoned “tsukudani” foods; processed livestock products such as canned livestock paste products and livestock meat hams and sausages; milk and dairy products such as processed milk, milk beverages, yogurt foods, fermented milk beverages, cheeses, ice creams, formulated milk powders, creams, and other dairy products; fats such as butters, margarines, and vegetable oils; basic seasonings such as soy sauces, misos, sauces, processed tomato seasonings, fermented seasoning “mirin” products, and vinegars; composite seasoning or food products such as cooking mixes, curry bases, sauces, dressings, noodle soup bases “mentsuyu”, spices, and other composite seasonings; frozen foods such as material frozen foods, half-cooked frozen foods, and cooked frozen food; confectioneries such as caramels, candies, chewing gums, chocolates, cookies, biscuits, cakes, pies, snacks, crackers, Japanese sweets, rice confectioneries, bean confectioneries, desserts, jellies, and other confectioneries; tasty beverages such as carbonated drinks, natural fruit juice, fruit juice drinks, fruit juice-containing soft drinks, fruit pulp drinks, granule-containing fruit drinks, vegetable-based beverages, soy milks, soy milk beverages, coffee beverages, tea beverages, powdered beverages, concentrated drinks, sports drinks, nutritional drinks, alcoholic drinks, and other taste beverages, other commercial foods such as baby food, dried seasoning powders “furikake”, and dried seasoning powders “ochazukenori”; infant formula milks; enteral nutrition foods; and functional foods (foods for specified health uses, nutritional functional foods, foods with function claims).

A preferred appearance of the food or drink composition according to the invention may for example be a supplement in a tablet form. In such a case, the amount of the active ingredient ingested and the calories ingested simultaneously with the active ingredient can precisely be known.

In addition, the preferred appearance of the food or drink composition according to the invention may for example be a liquid, especially a viscous liquid. The liquid can be spread throughout the entire inside of the upper respiratory tract, and the viscous liquid can be retained in the inside of the upper respiratory tract for a prolonged period, thereby allowing the effects to be exerted more efficiently.

Furthermore, the food or drink composition according to the invention can be provided or sold as a food or drink which claims its health use, such as use in removing foreign bodies from the upper respiratory tract, use in preventing foreign bodies from entering the upper respiratory tract, use in moisturizing the upper respiratory tract, use in inhibiting dryness of the upper respiratory tract mucosa, use in reducing the irritation of the mucosa of the upper respiratory tract, use in ameliorating the upper respiratory tract mucosal dryness, thirst, and scratchy throat of a smoker, and use in prophylaxis and/or therapy of cold symptoms.

The action of “claiming” includes every action for making a consumer to be informed of the aforementioned use, and any expression, which reminds the consumer of, or, which allows the consumer to assume, the aforementioned use is regarded as the action of “claiming” regardless of the purpose of the claiming, the contents of the claiming, or the claimed subjects or media.

The “claiming” is implemented preferably via an expression by which the consumer can recognize the aforementioned use directly. Those which may typically be exemplified include an activity to assign, deliver, display for the purpose of assignment or delivery and export a good or good package relating to a food or drink having a description of the aforementioned use thereon, as well as an activity to display or distribute advertisement materials, price lists, or transaction documents relating to goods having a description of the aforementioned use thereon or to provide such a detailed information also including a description of the aforementioned use therein via an electromagnetic method (such as internet).

Meanwhile, the claimed detail is preferably one which has been authorized by the relevant government (for example the claim was authorized under various regulations prescribed by the government and was implemented in a manner based on such an authorization). Such a claimed detail is preferably attached to the advertising materials at the site of selling such as a package, container, catalog, pamphlet, and POP as well as other documents.

The “claiming” may also be a claiming as a health food, functional food, enteral nutrition food, food for special use, food with health claims, food for specified health uses, food with function claims, nutritional functional food, and quasi-drug. Among these, one which may especially be exemplified is a claim authorized by Consumer Affairs Agency, for example, a claim authorized under the regulation on food for specified health uses or analogous regulations. Examples of the latter may be a claim as a food for specified health uses, a claim as a conditional food for specified health uses, a claim claiming an effect on body structure or function, and a disease risk reduction claim, and those exemplified more typically are a claim as a food for specified health uses (especially health use claim) prescribed under the Ordinance for Enforcement of Health Promotion Act (Ordinance of the Ministry of Health, Labor, and Welfare No.86 dated Apr. 30, 2003), a claim as a foods with function claims prescribed under Food Labeling Act (Act No.70 of 2013) and analogous claims.

### EXAMPLES

The present invention is further detailed in the following Examples. The present invention is not limited to these Examples.

### Production Example 1

In this Production Example, the agent for protecting an upper respiratory tract according to the present invention was produced. Each powder of 5 g of lactoferrin (manufactured by Morinaga Milk Industry Co., Ltd.), 0.5 g of lactoperoxidase (manufactured by Biopole), 4 g of glucose oxidase (manufactured by SHINNIHON CHEMICALS Corporation), 45 g of glucose (manufactured by NIHON SHOKUHIN KAKO CO., LTD.), 25.8 g of citric acid as a pH adjusting component (manufactured by San-Ei Gen F.F.I., Inc.), and 52.1 g of trisodium citrate (manufactured by San-Ei Gen F.F.I., Inc.), 150 g of erythritol (manufactured by Nikken Chemical Laboratory Co., Ltd.), 525 g of reduced maltose syrup (manufactured by TOWA KASEI CORPORATION), 300 g of sorbitol (manufactured by TOWA KASEI CORPORATION), 150 g of corn starch (manufactured by Oji Cornstarch Co., Ltd.), 7.5 g of acerola flavor (manufactured by Takasago International Corporation), 60g of sucrose fatty acid ester (manufactured by Mitsubishi-Chemical Foods Corporation), 180.1 g of xylitol (manufactured by TOWA KASEI CORPORATION) were combined and mixed uniformly, and a tableting machine (manufactured by HATA TEKKOSHO CO., LTD.) was used to compact the aforementioned mixed powder continuously under a pressure of 9.8 KPa at a tableting speed of 12 tablets per minutes to produce 1000 tablets each weighing 1.0 g. Each of these tablets contained 3.3 mg of the lactoferrin, 0.3 mg of the lactoperoxidase, 2.7 mg of the glucose oxidase, 30 mg of the glucose, and 51.8 mg of the pH adjusting component (total of citric acid and trisodium citrate).

### Test Example 1

### (1) Test Sample

In this Test Example, the agent for protecting an upper respiratory tract produced in Production Example 1 was used as a test sample to investigate the effect.

### (2) Testing Method

5 Healthy smokers of thirties to fifties (study subject: average years of past smoking of 18.4 years, average number of smoked cigarettes of 13.6/day) were included in the study, and each group was tested.
a) Non-treatment group (only smoking)

A study subject smoked a single cigarette within 10 minutes, and thereafter received none of test samples, and then 10 minutes after completion of the smoking, the study subject attended to an inquiry with regard to the mouth wetness, thirst, and scratchy throat, and be questioned for the following 3 points: “Is there any amelioration in mouth wetness?”, “Is there any inhibition in thirst?”, “Is there any inhibition in scratchy throat?”, which were judged by the subject as any of the following 4-grade scores: “Much”:2 points, “Moderate”:1 point, “Not so much”:-1 point, “None”:-2 points, and the relevant scores were analyzed statistically by a t-test.
b) Pre-smoking treatment group

A study subject before cigarette smoking was allowed preliminarily to take a single tablet of the test sample which was dissolved in saliva in the oral cavity within 10 minutes, after which a single cigarette was smoked within 10 minutes, and then 10 minutes after the completion of the smoking, the inquiry similar to that in the aforementioned a) was made to analyze each score statistically by the t-test.
c) Post-smoking treatment group

A study subject smoked a single cigarette within 10 minutes, and, after the completion of the smoking, received a single tablet of the test sample which was dissolved in saliva in the oral cavity within 10 minutes, and thereafter the inquiry similar to that in the aforementioned a) was made to analyze each score statistically by the t-test.

### (3) Results of Test

The results of this test are shown in Table 1 which includes scores in each group.

TABLE 1
Score in each group

| | Non- | | |
|---|---|---|---|
| | treatment | Pre- | Post- |
| | group | smoking | smoking |
| | (only | treatment | treatment |
| Status of test | smoking) | group | group |
| Is there any amelioration | −1.8 ± 0.4 | 0.6 ± 0.9* | 1.4 ± 0.5* |
| in mouth wetness? | | | |
| Is there any inhibition in | −1.6 ± 0.5 | −0.8 ± 1.1 | 0.6 ± 0.9* |
| thirst? | | | |
| Is there any inhibition in | −1.6 ± 0.5 | 0.2 ± 1.1* | −0.2 ± 1.6 |
| scratchy throat? | | | |

*Significant difference when compared with non-treatment group (p < 0.05)

As a result, the average score for “Is there any amelioration in mouth wetness?” in the only-smoking non-treatment group was −1.8 points, showing no increase in the mouth wetness when compared with that before smoking.

On the other hand, the pre-smoking treatment group had 0.6 points and the post-smoking treatment group had 1.4 points, showing that any of the groups of the pre-smoking treatment and the post-smoking treatment exhibited a significant increase in the upper respiratory tract wetness when compared with the only-smoking non-treatment group.

The average score for “Is there any inhibition in thirst?” in the only-smoking non-treatment group was −1.6 points, showing no reduction in the thirst.

On the other hand, the pre-smoking treatment group had −0.8 points and the post-smoking treatment group had 0.6 points, showing that the post-smoking treatment group exhibited a significant thirst inhibition when compared with the only-smoking non-treatment group.

The average score for “Is there any inhibition in scratchy throat?” in the only-smoking non-treatment group was −1.6 points, showing no reduction in the scratchy throat.

On the other hand, the pre-smoking treatment group had 0.2 points and the post-smoking treatment group had −0.2 points, showing that the pre-smoking treatment group exhibited a significant scratchy throat inhibition when compared with the only-smoking non-treatment group.

The components consisting of the lactoferrin, the lactoperoxidase, the glucose oxidase, the glucose, and the citric acid and trisodium citrate as pH adjusting components in the production of the agent for protecting an upper respiratory tract in the aforementioned Production Example 1 was substituted by 132.4 g of corn starch to produce a comparative sample, and this comparative sample was subjected to the test similar to that in the pre-smoking treatment group in the aforementioned b).

As a result, the comparative sample in the pre-smoking treatment group exhibited the scores almost equivalent to those in the only-smoking non-treatment group, showing no inhibitory effects on the upper respiratory tract mucosa dryness.

Thus, it was evident that the agent for protecting an upper respiratory tract according to the present invention, even when administered before smoking, has the effects to ameliorate the mouth wetness and inhibit the scratchy throat. While the upper respiratory tract mucosal dryness and the scratchy throat associated with smoking are remedied generally for example by ingesting a throat lozenge after smoking in an attempt to ameliorate the symptoms, it became evident that the agent for protecting an upper respiratory tract according to the present invention can exert its effect to enable the prophylaxis of such upper respiratory tract mucosal dryness and scratchy throat via preliminary administration before smoking.

Based on these results, it became evident that the agent for protecting an upper respiratory tract according to the present invention can inhibit the reduction in the wetness in the oral cavity associated with smoking when administered either before or after smoking, can inhibit the thirst associated with smoking when administered after smoking, and can inhibit the scratchy throat associated with smoking when administered before smoking. Accordingly, it was suggested that the agent for protecting an upper respiratory tract according to the present invention reduces the irritation to the mucosa of the oral cavity, pharynx, and larynx and prevents the mucosa from being dried.

### Test Example 2

### (1) Test Sample

This test investigated the cold symptoms inhibiting effect of the ingestion of a pharynx protection agent of the present invention while comparing with the effects of gargle habit or mask-wearing habit.

In this Test Example, the agent for protecting an upper respiratory tract produced in Production Example 1 was used as a test sample similarly to Test Example 1, and its effects were investigated.

### (2) Testing Method

Healthy adults giving their consent after being informed of the purpose and the detail of the test were included as study subjects. First, a background surveillance was made for gargle habit and mask-wearing habit. Then, the study subjects were assigned randomly into 2 groups, and the test group ingested 3 tablets a day of the test sample during the time of traveling, going out, commuting, and the like by dissolving the tablet with saliva without chewing, while the control group did not ingest the tablets.

Presence or absence of the cold symptoms including fever (recorded body temperature (° C.) upon suspected fever), sore throat, cough, nasal discharge, nasal congestion, phlegm, headache, and joint pain/muscle pain was recorded in a diary on the symptom basis, and if there was any record, the cold symptoms were regarded to be present on the relevant day. A statistic analysis was made for the presence or absence of the cold symptoms development by Fisher's exact test and for the number of days of development by Wilcoxon rank sum test. Subpopulations were also assigned on the basis of background factors including gargle habit and mask-wearing habit, and the subpopulation analysis was made.

### (3) Results of Test

The results of this test are indicated in Tables 2 to 6 shown below.

TABLE 2
Background factor

| | Test group | Control group | |
|---|---|---|---|
| Item | n = 129 | n = 136 | P value |
| Gargle habit | 69:60 | 89:47 | 0.0601 |
| (present:absent) | (53.5%) | (65.4%) | |
| Mask-wearing | 35:94 | 40:96 | 0.6852 |
| habit | (27.1%) | (29.4%) | |
| (present:absent) | | | |

% cases having background factor

TABLE 3
Presence or absence of cold symptoms

| | | Group | | |
|---|---|---|---|---|
| | | Test group | Control group | |
| | | n = 129 | n = 136 | P value |
| Subpopulation | Gargle habit | 23:46 | 24:65 | 0.4831 |
| | present | (33.3%) | (27.0%) | |
| | n = 158 | | | |
| | Gargle habit | 21:39 | 25:22 | 0.0771 |
| | absent | (35.0%) | (53.2%) | |
| | n = 107 | | | |

| | | |
|---|---|---|
| P value | 0.8545 | 0.0045 |

Present:Absent

TABLE 4
Number of cold symptoms days

| | | Group | | |
|---|---|---|---|---|
| | | Test group | Control group | |
| | | n = 129 | n = 136 | P value |
| Subpopulation | Gargle habit | 1.9 ± 4.8 | 1.8 ± 4.0 | 0.6011 |
| | present | | | |
| | n = 158 | | | |
| | Gargle habit | 2.4 ± 5.0 | 3.3 ± 4.7 | 0.0612 |
| | absent | | | |
| | n = 107 | | | |

| | | |
|---|---|---|
| P value | 0.7777 | 0.0051 |

Average ± Standard deviation

TABLE 5
Presence or absence of fever of 38° C. or higher

| | | Group | | |
|---|---|---|---|---|
| | | Test group | Control group | |
| | | n = 129 | n = 136 | P value |
| Subpopulation | Mask-wearing | 1:34 | 0:40 | 0.4667 |
| | habit | (2.9%) | (0.0%) | |
| | present | | | |
| | n = 75 | | | |
| | Mask-wearing | 0:94 | 4:92 | 0.1211 |
| | habit | (0.0%) | (4.2%) | |
| | absent | | | |
| | n = 190 | | | |

| | | |
|---|---|---|
| P value | 0.2713 | 0.3199 |

Present:Absent

TABLE 6
Number of days of fever of 38° C. or higher

| | | Group | | |
|---|---|---|---|---|
| | | Test group | Control group | |
| | | n = 129 | n = 136 | P value |
| Subpopulation | Mask-wearing | 0.1 ± 0.5 | 0.0 ± 0.0 | 0.2973 |
| | habit | | | |
| | present | | | |
| | n = 75 | | | |
| | Mask-wearing | 0.0 ± 0.0 | 0.1 ± 0.4 | 0.0466 |
| | habit | | | |
| | absent | | | |
| | n = 190 | | | |

| | | |
|---|---|---|
| P value | 0.1049 | 0.1945 |

Average ± Standard deviation

As a result of analysis of 265 test-completing cases (129 cases in the test group, 136 cases in the control group), an inter-group disproportion was observed in the ratio of the cases with gargle habit among the background factors (Table 2, P<0.1). On the other hand, no inter-group disproportion was observed in the ratio of the cases with mask-wearing habit among the background factors (Table 2).

In 158 subpopulation cases with gargle habit, no inter-group difference was observed in the presence or absence of the cold symptoms (test group, 23:46 (33.3%), control group, 24:65 (27.0%)) or in the number of symptomatic days (test group, 1.9±4.8 days, control group, 1.8±4.0 days) (Table 3, 4). On the other hand, in 107 subpopulation cases without gargle habit, the test group exhibited a tendency of reduction in the presence or absence of cold symptoms (test group, 21:39 (35.0%), control group, 25:22 (53.2%)) and in the number of symptomatic days (test group, 2.4±5.0 days, control group,3.3±4.7 days)(P<0.1).

In 136 control group cases, the subpopulation without gargle habit exhibited a significant increase when compared with the subpopulation with gargle habit in the presence or absence of cold symptoms (subpopulation with gargle habit, 24:65 (27.0%), subpopulation without gargle habit, 25:22 (53.2%)) and in the number of symptomatic days (subpopulation with gargle habit, 1.8±4.0 days, subpopulation without gargle habit, 3.3±4.7 days) (P<0.05). On the other hand, 129 test group cases exhibited no difference between the subpopulations with and without gargle habit in the presence or absence of cold symptoms (subpopulation with gargle habit, 23:46 (33.3%), subpopulation without gargle habit, 21:39 (35.0%)) or in the number of symptomatic days (subpopulation with gargle habit, 1.9±4.8 days, subpopulation without gargle habit, 2.4±5.0 days).

Thus, the cold symptoms risk is increased in the control group without gargle habit while the cold symptoms risk is not changed in the test group even without gargle habit, it was proven that the pharynx protection agent of the present invention has an effect to reduce the cold symptoms risk.

In 75 subpopulation cases with mask-wearing habit, no inter-group difference was observed in the presence or absence of fever of 38° C. or higher (test group, 1:34 (2.9%), control group, 0:40 (0.0%)) or in the number of symptomatic days (test group, 0.1±0.5 days, control group, 0.0±0.0 days) (Tables 5 and 6). On the other hand, 190 subpopulation cases without mask-wearing habit exhibited no difference between the groups in the presence or absence of fever of 38° C. or higher (test group, 0:94 (0.0%), control group, 4:92 (4.2%)), while the number of days of fever of 38° C. or higher was lower significantly in the test group when compared with the control group showed (Table 6, test group, 0.0+0.0 days, control group, 0.1±0.4 days, P<0.05). Accordingly, it was proven that, in subpopulation without mask-wearing habit, the pharynx protection agent of the present invention has the effect to inhibit the exacerbation of the cold symptoms to a serious disease.

In 136 control group cases, there was no significant difference between the subpopulation with mask-wearing habit and the subpopulation without mask-wearing habit in the presence or absence of fever of 38° C. or higher (subpopulation with mask-wearing habit, 0:40 (0.0%), subpopulation without mask-wearing habit, 4:92 (4.2%)) or in the number of symptomatic days (subpopulation with mask-wearing habit, 0.0±0.0 days, subpopulation without mask-wearing habit, 0.1±0.4 days). In 129 test group cases, there is no difference between the subpopulations with and without mask-wearing habit in the presence or absence of fever of 38° C. or higher (subpopulation with mask-wearing habit, 1:34 (2.9%), subpopulation without mask-wearing habit, 0:94 (0.0%)) or in the number of symptomatic days (subpopulation with mask-wearing habit, 0.1±0.5 days, subpopulation without mask-wearing habit, 0.0±0.0 days). Accordingly, it became evident that the risk of fever of 38° C. or higher is not different between the control group and the test group even without mask-wearing habit.

Based on the results described above, it was suggested that the ingestion of the agent for protecting an upper respiratory tract according to the present invention can substitute prophylactic means such as gargle or mask-wearing the use of which is impossible.

### INDUSTRIAL APPLICABILITY

The agent for protecting an upper respiratory tract according to the present invention and the food or drink composition for protecting an upper respiratory tract can ameliorate various symptoms relating to upper respiratory tracts.

Typically, the agent for protecting an upper respiratory tract according to the present invention and the food or drink composition for protecting an upper respiratory tract can reduce the discomforts associated with smoking such as upper respiratory tract mucosal dryness, thirst, and scratchy throat and also enables a convenient administration because it has reduced side effects even if given for a prolonged period.

In addition, the agent for protecting an upper respiratory tract according to the present invention and the food or drink composition for protecting an upper respiratory tract have inhibitory effects on the cold symptoms development, the number of symptomatic days, and the exacerbation to a serious disease via upper respiratory tract protection. Moreover, the present invention allows the ingestion volume and the number of ingestion times to be selected arbitrarily because of its reduced side effect and high safety, thereby enabling a long term routine ingestion.

Moreover, the present invention can employ the following constitutions.
[1] An agent for protecting an upper respiratory tract containing lactoferrin, lactoperoxidase, glucose oxidase, glucose source, and pH adjusting component as active ingredients.
[2] The agent for protecting the upper respiratory tract described in [1] wherein the aforementioned upper respiratory tract protection is based on at least one selected from removal of foreign bodies from the upper respiratory tract, prevention of foreign bodies from entering the upper respiratory tract, moisturization of the upper respiratory tract, inhibition of the dryness of the mucosa of the upper respiratory tract, and reduction in the irritation of the mucosa of the upper respiratory tract.
[3] The agent for protecting the upper respiratory tract described in [1] or
[2] wherein the aforementioned upper respiratory tract protection is prophylaxis and/or therapy of cold symptom.
[4] The agent for protecting the upper respiratory tract described in [3] wherein the at least one cold symptom is symptoms selected from the group consisting of fever, sore throat, cough, nasal discharge, nasal congestion, phlegm, headache, joint pain, and muscle pain.
[5] The agent for protecting the upper respiratory tract described in [3] or [4] wherein the aforementioned cold symptom is a symptom of a high body temperature of 38° C. or higher.
[6] The agent for protecting the upper respiratory tract described in [1] or [2] used by smokers.
[7] The agent for protecting the upper respiratory tract described in [6] used before smoking.
[8] The agent for protecting the upper respiratory tract described in any of [1] to [7] wherein the aforementioned upper respiratory tract is a pharynx or larynx.
[9] The agent for protecting the upper respiratory tract described in any of [1] to [8] wherein the aforementioned pH adjusting component is an organic acid and/or a salt of the organic acid.
[10] The agent for protecting the upper respiratory tract described in [9] wherein the aforementioned organic acid is one or more organic acids selected from the group consisting of citric acid, lactic acid, malic acid, succinic acid, tartaric acid, and glutamic acid.
[11] A food or drink composition for protecting an upper respiratory tract containing lactoferrin, lactoperoxidase, glucose oxidase, glucose source, and a pH adjusting component as active ingredients.
[12] Use of lactoferrin, lactoperoxidase, glucose oxidase, glucose source, and a pH adjusting component for an agent for protecting an upper respiratory tract.
[13] Use of lactoferrin, lactoperoxidase, glucose oxidase, glucose source, and a pH adjusting component for a food or drink composition for protecting an upper respiratory tract.
[14] Use of lactoferrin, lactoperoxidase, glucose oxidase, glucose source, and a pH adjusting component for producing an agent for protecting an upper respiratory tract.
[15] Use of lactoferrin, lactoperoxidase, glucose oxidase, glucose source, and a pH adjusting component for producing a food or drink composition for protecting an upper respiratory tract.
[16] Use of lactoferrin, lactoperoxidase, glucose oxidase, glucose source, and a pH adjusting component in an agent for protecting an upper respiratory tract.
[17] Use of lactoferrin, lactoperoxidase, glucose oxidase, glucose source, and a pH adjusting component in a food or drink composition for protecting an upper respiratory tract.
[18] Lactoferrin, lactoperoxidase, glucose oxidase, glucose source, and a pH adjusting component for use in prophylaxis, amelioration, and/or therapy of an upper respiratory tract disorder.
[19] A method for prophylaxis, amelioration, and/or therapy of an upper respiratory tract disorder using lactoferrin, lactoperoxidase, glucose oxidase, glucose source, and a pH adjusting component as active ingredients.

1. An agent for protecting an upper respiratory tract containing lactoferrin, lactoperoxidase, glucose oxidase, glucose source, and a pH adjusting component as active ingredients.

2. The agent for protecting the upper respiratory tract according to claim 1 wherein the upper respiratory tract protection is based on at least one selected from removal of foreign bodies from the upper respiratory tract, prevention of foreign bodies from entering the upper respiratory tract, moisturization of the upper respiratory tract, inhibition of the dryness of the mucosa of the upper respiratory tract, and reduction in the irritation of the mucosa of the upper respiratory tract.

3. The agent for protecting the upper respiratory tract according to claim 1, wherein the upper respiratory tract protection is prophylaxis and/or therapy of a cold symptom.

4. The agent for protecting the upper respiratory tract according to claim 3 wherein the at least one cold symptom is symptoms selected from the group consisting of fever, sore throat, cough, nasal discharge, nasal congestion, phlegm, headache, joint pain, and muscle pain.

5. The agent for protecting the upper respiratory tract according to claim 3, wherein the cold symptom is a symptom of a high body temperature of 38° C. or higher.

6. The agent for protecting the upper respiratory tract according to claim 1, used by smokers.

7. The agent for protecting the upper respiratory tract according to claim 6 used before smoking.

8. The agent for protecting the upper respiratory tract according to claim 1 wherein the upper respiratory tract is a pharynx or larynx.

9. The agent for protecting the upper respiratory tract according to claim 1 wherein the pH adjusting component is an organic acid and/or a salt of the organic acid.

10. The agent for protecting the upper respiratory tract according to claim 9 wherein the organic acid is one or more organic acids selected from the group consisting of citric acid, lactic acid, malic acid, succinic acid, tartaric acid, and glutamic acid.

11. A food or drink composition for protecting an upper respiratory tract comprising the agent according to claim 1 containing lactoferrin, lactoperoxidase, glucose oxidase, glucose source, and a pH adjusting component as active ingredients.

12. Use of the agent according to claim 1 comprising lactoferrin, lactoperoxidase, glucose oxidase, glucose source, and a pH adjusting component for an agent for protecting an upper respiratory tract.

13. Use of the agent according to claim 1 comprising lactoferrin, lactoperoxidase, glucose oxidase, glucose source, and a pH adjusting component for a food or drink composition for protecting an upper respiratory tract.

14. Use of the agent according to claim 1 comprising lactoferrin, lactoperoxidase, glucose oxidase, glucose source, and a pH adjusting component for producing an agent for protecting an upper respiratory tract.

15. Use of the agent according to claim 1 comprising lactoferrin, lactoperoxidase, glucose oxidase, glucose source, and a pH adjusting component for producing a food or drink composition for protecting an upper respiratory tract.

16. Use of the agent according to claim 1 comprising lactoferrin, lactoperoxidase, glucose oxidase, glucose source, and a pH adjusting component in an agent for protecting an upper respiratory tract.

17. Use of the agent according to claim 1 comprising lactoferrin, lactoperoxidase, glucose oxidase, glucose source, and a pH adjusting component in a food or drink composition for protecting an upper respiratory tract.

18. Lactoferrin, lactoperoxidase, glucose oxidase, glucose source, and a pH adjusting component for use in prophylaxis, amelioration, and/or therapy of an upper respiratory tract disorder.

19. A method for prophylaxis, amelioration, and/or therapy of an upper respiratory tract disorder using lactoferrin, lactoperoxidase, glucose oxidase, glucose source, and a pH adjusting component as active ingredients,
